(19) United States
(12) Patent Application Publication
(10) Pub. No.: US 2009/0227992 A1
(43) Pub. Date: Sep. 10, 2009

(54) Shock-Wave Generating Device, Such as for the Treatment of Calcific Aortic Stenosis

(75) Inventors: Yael Nir, Caesarea (IL); Moshe Y. Flugelman, Haifa (IL)

Correspondence Address: MARTIN D. MOYNIHAN d/b/a PRTSI, INC., P.O. BOX 16446, ARLINGTON, VA, 22215, US

(73) Assignee: ReLeaf Medical Ltd, Kiryat-Shmona (IL)

(21) Appl. No.: 12/223,458

(22) PCT Filed: Feb. 1, 2007
(86) PCT No.: PCT/IL07/00135

§ 371 Date: Nov. 21, 2008

Related U.S. Application Data
(60) Provisional application No. 60/764,343, filed on Feb. 2, 2006.

(51) Int. Cl. A61B 18/20

(52) U.S. Cl. 606/7

(57) ABSTRACT

Disclosed is a method for removing deposits from an in vivo tissue, such as removing calcified plaque from a calcified aortic valve by removing the deposits with the help of shock waves produced by a light-source such as a laser in an aqueous liquid in proximity of the tissue as well as a shock-wave generating device useful in implementing the method. Disclosed is a minimally invasive valve-sparing treatment of calcific aortic stenosis.

### FIELD AND BACKGROUND OF THE INVENTION

The present invention relates to shock wave-generating devices useful in the field of medicine. The present invention also relates to the removal of deposits such as sediment, plaque, debris or encrustations from in vivo surfaces or tissues with the help of shock waves. The present invention also relates to cardiovascular medicine and more particularly to a valve-sparing treatment of calcific aortic stenosis.

Calcific aortic stenosis accounts for the vast majority of aortic valve disease. In calcific aortic stenosis there is a progressive deposition of a calcified plaque comprising a major collagen component containing hard nodules of calcium phosphate and/or hydroxyapatite on the aortic face of the aortic valve cusps. As the relatively inflexible layer of calcified plaque thickens, systolic opening of the affected valve is increasingly restricted (typically at a rate of 0.1 cm²/year), reducing the aortic valve area to less than 1.2 cm² in moderate cases and to less than 1.0 cm² in severe cases. The pressure gradient across a stenotic valve will typically rise to about 3-4 ms⁻¹ jet velocity in moderate and to over 4 ms⁻¹ in severe cases. Eventually the valve cusps fuse together. Consequently, calcific aortic stenosis often leads to reduced systemic blood flow, increased cardiac stress and chronic left ventricular pressure overloading.

Attempts at valve-sparing procedures for treating calcific aortic stenosis, for example by mechanical debridement of the calcified plaque, have been unsuccessful as damage to the cusps often leads to aortic restenosis or valvular insufficiency caused by cusp scarring and retraction.

As a result, the only currently accepted treatment of severe symptomatic calcific aortic stenosis is valve replacement, for example with a prosthetic or biological valve. As is well-known to one skilled in the art, valve replacement requires dangerous open heart surgery followed by chronic administration of anticoagulants (e.g., warfarin) and/or subsequent valve replacements every few years. A person having undergone valve replacement will have an increased risk of thromboembolisms, endocarditis and bleeding related to chronic treatment with anticoagulants.

Isner J M, Michlewitz H, Clarke R H, Donaldson R F, Konstam M A, Salem D N in Am Heart J 1985, 109(3), 448-452 teach excision of calcified plaque from aortic valve cusps using 500 millisecond pulses of 10.6 μm light from a CO₂ laser at 3 W and at 3.8 W. The laser was used like a knife to cut through the calcified plaque which was then mechanically peeled away as a sheet. Use of the laser provided no advantage over mechanical debridement.

Attempts have been made to use laser photoablation or sonic ablation as alternatives to mechanical debridement in valve-sparing procedures to remove calcified plaque from aortic valves.

Williamson W A, Aretz A T, Weng G, Shahian D M, Hamilton W M, Pankratov M M and Shapshay S M in Lasers in Surgery and Medicine 1993, 13, 421-428 teach the removal of calcified plaque from dry (i.e., not immersed in a liquid) aortic valve cusps using Er:YSG lasers, Ho:YAG lasers and a blunt-probe ultrasonic cleaner (Cavitron™ by DENTSPLY International, York, Pa., USA). The ultrasonic cleaner shattered, disrupted and delaminated the cusps. The Ho:YAG laser was used to generate 200 μs pulses of 2.12 μm light at 0.6 J/pulse light at 6 Hz (fluence of 11.75 J cm⁻²) that were focused with a lens to illuminate a spot of 300 μm on the cusps for direct photoablation of the calcium concretions but was found to be ineffective in removing the concretions and caused thermal damage to the cusps. The Er:YSGG laser was used to generate 250 μs pulses of 2.79 μm light at 0.2 J/pulse at 6 Hz (fluence of 238 J cm⁻²) guided through a 300 μm diameter core optical fiber for direct photoablation of the calcium concretions. Although more effective than the Ho:YAG laser, the Er:YSGG laser lead to thermal damage and disruption of cusp tissue by shockwaves generated by the photoacoustic effect in the concretions.

The photoacoustic effect is a well known effect whereby a sufficiently high amount of light energy (usually produced by a laser) is absorbed by a sufficiently small volume of material so that the material explosively vaporizes. If the vaporization is in a liquid, the vapor of material produces an expanding bubble. When the energy of the vapor dissipates the bubble collapses, which under certain conditions produces a second shock wave.

It is known to use photoacoustically-generated shockwaves in the field of medicine.

Laser lithotripters are known in the art and include endoscopically mounted lasers to photoacoustically generate shockwaves for destroying isolated concretions such as mineral salt calculi of the kidney, ureter, bladder, or gallbladder. Such lithotripters are described, for example, in U.S. Pat. Nos. 5,059,200; 5,242,454; 5,496,306; 6,375,651; 6,726,681 and 7,104,983. Such lithotripters irradiate a concretion with laser pulses until the concretion is destroyed by a combination of photoablation and shockwaves generated photoacoustically in the concretion, optionally in the plume of debris generated by photoablation of the concretion, see U.S. Pat. No. 5,496,306 and optionally in the fluid in the vicinity of the concretion see U.S. Pat. No. 5,059,200.

Laser powered ultrasonic transducers configured to irradiate a fluid with a train of laser pulses at ultrasonic frequencies so as to generate shock waves at ultrasonic frequencies through the photoacoustic effect are known in the art for clearing soft occlusions such as blood clots, thrombi and atherosclerotic plaque from the inside of blood vessels. Such transducers are described, for example, in U.S. Pat. Nos. 6,022,309 and 6,428,531.

In U.S. Pat. No. 6,517,531 and is taught a suction device suitable for use as a component of a laser lithotripsy endoscope to collect fragments and debris from an eroded concretion.

It would be highly advantageous to have a method for treating calcific aortic stenosis that does not involve replacing or damaging the aortic valve. More generally, it would be highly advantageous to have a method for removing deposits from in vivo tissue without damaging the tissue.

### SUMMARY OF THE INVENTION

The present invention is of methods and devices for removing deposits from in vivo tissue or surface without damaging the functionality of the tissue or surface, in embodiments for removing calcified plaque from an aortic valve. Embodiments of the present invention provide for removal of the deposits under low visibility conditions and under conditions where there is a flow of liquid, such as blood, past the tissue.

According to the teachings of the present invention there is provided a method for removing deposits from an in vivo tissue comprising: a) providing a probe having a distal end and a distal tip which comprises a light-guide, a distal tip of the light-guide in proximity of a distal end of the probe; and b) for a plurality of areas of in vivo tissue of a subject (in embodiments a human, in embodiments a cadaver, in embodiments a non-human animal) the tissue covered at least in part by a deposits wherein the areas are immersed in an aqueous liquid (e.g., blood or other bodily fluid):
i) placing the distal tip of the light-guide at a distance from an area from the plurality of areas of the tissue;
ii) through the light-guide guiding a pulse train which comprises at least one pulse of light, each pulse of light of an energy, of a duration less than about 300 ns and including at least one wavelength of light such as to generate a shock wave in the aqueous liquid at an interface between the distal tip of the light-guide and the aqueous liquid, the pulse train sufficient to dislodge at least some of the deposits from the area; and
iii) repeating i and ii at least once, optionally at a different area from the group of areas
   thereby removing at least some of the deposits from the tissue, wherein at least one wavelength is a wavelength having a water absorption coefficient of no less than about 10³ cm⁻¹ selected from the group consisting of between 2.8 μm and 3.5 μm, between 5.9 μm and 6.4 μm and between 12 μm and 18 μm. Suitable guides include, but are not limited to, catheters, endoscopes, arthroscopes, amnioscopes, esophagogastroduodenoscopes, cystoscopes, colonoscopes, pelviscopes, bronchoscopes, laparscopes, ureteroscopes and nephroscopes. Typical deposits removed include adhesions, calcifications, concretions, debris, encrustations, plaque, calcified plaque and sediment. Typical tissues include, but are not limited to, a luminal surface of a vein, a luminal surface of an artery, a heart valve, an aortic valve, a cardiac valve seat, especially prior to implantation of an artificial valve or alternate valve as well as non-cardiovascular tissue such as gastric mucosa, small bowel and colonic mucosa, peritoneum, adhesions, ligaments, luminal surface of ureters, fasciae, articular discs and sinovial tissues.

In embodiments, the distal tip of the probe protrudes a distance from the distal tip of the light-guide and the placing the distal tip of the light-guide at a distance from the area to be treated (as described above) comprises contacting the distal tip of the probe against tissue in proximity of the area.

More specifically, according to the teachings of the present invention there is also provided a valve-sparing method for treatment of calcific aortic stenosis comprising: a) providing a cardiac catheter having a distal end and a distal tip which comprises a light-guide, a distal tip of the light-guide in proximity of the distal end of the catheter; and for b) for a plurality of areas of a calcified aortic valve of a heart of a subject (in embodiments a human, in embodiments a cadaver, in embodiments a non-human animal) wherein the calcified aortic valve is immersed in an aqueous liquid (e.g., blood or other bodily fluid):
i) placing the distal tip of the light-guide at a distance from an area from the plurality of areas of the calcified aortic valve;
ii) through the light-guide guiding a pulse train which comprises at least one pulse of light, each pulse of light of an energy, of a duration less than about 300 ns and including at least one wavelength of light such as to generate a shock wave in the aqueous liquid at an interface between the distal tip of the light-guide and the aqueous liquid, the pulse train sufficient to dislodge at least some plaque coating the area; and
iii) repeating i and ii at least once, optionally at a different area from the group of areas
   thereby treating the calcified aortic valve, wherein at least one wavelength is a wavelength having a water absorption coefficient of no less than about 10³ cm⁻¹ selected from the group consisting of between 2.8 μm and 3.5 μm, between 5.9 μm and 6.4 μm and between 12 μm and 18 μm. In embodiments, the heart is beating. In embodiments, generation of the pulse train of light is coordinated with the beating of the heart. In embodiments, the heart beats at a rate of at least about 100 beats per minute, at least about 180 beats per minute and even at least about 200 beats per minute.

In embodiments, the distal tip of the catheter protrudes a distance from the distal tip of the light-guide and the placing the distal tip of the light-guide at a distance from the area to be treated (as described above) comprises contacting the distal tip of the catheter against the aortic valve in proximity of the area.

In embodiments of methods of the present invention, the distance from the distal tip of the light-guide and the area treated is not greater than about 2 mm and even not greater than about 1 mm. In embodiments of the present invention, the distance from the distal tip of the light-guide and the area treated is not less than about 0.3 mm and even not less than about 0.5 mm.

In embodiments of methods of the present invention, substantially all wavelengths of light guided through the light-guide have a water absorption coefficient of no less than about 10³ cm⁻¹, see FIG. 1. In embodiments of methods of the present invention, substantially all wavelengths of light guided through the light-guide have a water absorption coefficient of no less than 10⁴ cm⁻¹, see FIG. 1. In embodiments, at least one wavelength used to generate a shockwave is about 2.94 μm.

In embodiments of methods of the present invention, the duration of the pulse of light is less than about 200 ns, less than about 100 ns, less than about 50 ns and even less than about 10 ns.

In embodiments of methods of the present invention, the interface of the light-guide and the aqueous liquid is not less than about 0.008 mm² (equivalent to the area of a 100 μm diameter circle), not less than about 0.03 mm² (equivalent to the area of a 200 μm diameter circle), and even not less than about 0.126 mm² (equivalent to the area of a 400 μm diameter circle),

In embodiments of methods of the present invention, the light-guide comprises at least one substantially circular cross section optical fiber with a diameter of not less than 100 μm, not less than about 200 μm, and even not less than about 400 μm. In preferred embodiments, the diameter is between about 400 μm and about 600 μm.

In embodiments of methods of the present invention, the energy emerging from the distal tip of the light-guide is at least about 1 mJ, at least about 2 mJ and even at least about 5 mJ.

In embodiments of methods of the present invention, the energy emerging from the distal tip of the light-guide is less than about 1000 mJ, less than about 500 mJ and even less than about 100 mJ.

In embodiments of methods of the present invention, a succeeding pulse of light is initiated after complete collapse of a bubble produced by a preceding pulse of light.

In embodiments of methods of the present invention, the pulse train comprises not less than two pulses of light at a frequency of not lower than about 2 Hz.

In embodiments of methods of the present invention, the pulse train comprises not less than about five pulses of light.

In embodiments of methods of the present invention, the frequency of the pulse train is not lower than 5 Hz and even not lower than about 10 Hz. In embodiments of methods of the present invention, the frequency is not higher than about 1000 Hz, not higher than about 500 Hz, not higher than about 250 Hz, and even not higher than about 100 Hz.

In embodiments of the method of the present invention, the pulse train of light is produced by a laser. In embodiments of methods of the present invention, the pulse-train of light is produced by a Q-switched Er:YAG laser, for example configured to produce pulses of 2.94 μm light having a duration of between 100 and 200 ns. In embodiments of methods of the present invention, the pulse-train of light is produced by an Nd:YAG laser-pumped OPO, for example configured to produce pulses of 2.94 μm light having a duration of between 1 and 15 ns.

In embodiments of methods of the present invention, iv) prior to ii (generation of shock waves with the help of the light), the area is observed. In embodiments, observing the area comprises optically observing the area. In embodiments, the optical observation is through the light-guide or through another light guide associated with the probe or catheter different from the light-guide. In embodiments, during the optical observation, the area is illuminated with at least one wavelength of light that is not substantially absorbed by the aqueous liquid. In embodiments, the illuminating is through the light-guide, through another light guide associated with the probe or with the catheter different from the light-guide, or through a fluid conduit associated with the catheter or the probe.

In embodiments of methods of the present invention, v) prior to the optical observing, the area is irrigated with a substantially transparent liquid (e.g., saline solution) so as to displace the aqueous liquid to allow clearer the observation of the area. In embodiments of methods of the present invention, the irrigating is through an outlet of a fluid conduit associated with the catheter or the probe in proximity of the distal end of the catheter or of the probe. In embodiments, the irrigation is intermittent. In embodiments, the irrigation is continuous.

In embodiments of methods of the present invention, c) during b (the production of shock waves), an emboli trap is deployed downstream of the area subject to the shock waves so as to trap at least some of the dislodged plaque.

In embodiments, the methods of the present invention further comprise vi) aspirating at least some of the dislodged plaque. In embodiments, the aspiration is intermittent. In embodiments, the aspiration is continuous. In embodiments, the aspiration occurs during the guiding of the pulse train of the light. In embodiments, the aspiration occurs through an inlet of an aspiration fluid conduit associated with the catheter or the probe, the inlet located in proximity of the distal end of the catheter or the probe.

In embodiments of the methods of the present invention, the methods further comprise: d) cutting commissural fusion of a calcified aortic valve, in embodiments prior to removal of plaque, in embodiments subsequent to removal of plaque. In embodiments, the cutting is with light projected through a light guide associated with the catheter or probe. In embodiments, the cutting is with light projected through the light-guide. In embodiments, the cutting is with light projected through a light guide different from the light-guide through which the shock wave producing light is guided.

In embodiments, the methods of the present invention further comprise: e) administering an anti-scarring agent (e.g., Pacxitacel) to at least part of an aortic valve. In embodiments, the administering is through a conduit in the catheter or the probe. In embodiments, the part of the aortic valve is a cusp of the aortic valve.

According to the teachings of the present invention there is also provided a shock-wave generating device suitable for producing in vivo shock waves suitable for the removal of deposits (such as calcified plaque) from a tissue (such as an aortic valve), comprising:
a) a probe with a distal end, a distal tip and a proximal end, which comprises:
   i) a light-guide configured to guide shock wave-generating light from a proximal end of the light-guide to a distal tip of the light-guide;
b) a light-source functionally associated with the probe so as to direct light into the light-guide from the proximal end, the light-source configured to generate a pulse train which comprises at least one pulse of light, each pulse of light of an energy, of a duration less than about 300 ns and including at least one wavelength of light such as to generate a shock wave in an aqueous liquid
   wherein at least one of the wavelengths is a wavelength having a water absorption coefficient of no less than about 10³ cm⁻¹ selected from the group consisting of between 2.8 μm and 3.5 μm, between 5.9 μm and 6.4 μm and between 12 μm and 18 μm. In embodiments, at least one the wavelength produced by the light-source is 2.94 μm. In embodiments, substantially all wavelengths of light guided through the light-guide have a water absorption coefficient of no less than about 10³ cm⁻¹ and in embodiments even of no less than 10⁴ cm⁻¹, e.g., the light-source produces only such light or there is a filter or the like that prevents light having a lower absorption coefficient from passing through the light-guide.

In embodiments, the duration of the pulses of light is less than about 200 ns, less than about 100 ns, less than 50 ns and even less than 10 ns.

In embodiments, the device is configured so that a pulse of light generated by the light-source and guided through the light-guide emerges from the distal tip of the light-guide at an energy of at least about 0.5 mJ, at least about 1 mJ, at least about 2 mJ, and even at least about 5 mJ.

In embodiments, the pulse train which comprises of not less than two pulses of light at a frequency of not lower than 2 Hz. In embodiments the pulse train which comprises of not less than five pulses of light. In embodiments, the frequency is not lower than 5 Hz and even not lower than 10 Hz. In embodiments, the frequency is not higher than 1000 Hz, not higher than 500 Hz, not higher than 250 Hz and even not higher than 100 Hz.

In embodiments the light source is a laser. In embodiments, the light-source is a Q-switched Er:YAG laser configured to produce pulses of 2.94 μm light having a duration of between about 100 and 200 ns. In embodiments, the light-source is a Nd:YAG laser-pumped OPO configured to produce pulses of 2.94 μm light having a duration of between about 1 and 15 ns.

In embodiments, the distal tip of the probe protrudes a distance from the distal tip of the light-guide. In embodiments, the distance not less than about 0.3 mm and even not less than about 0.5 mm. In embodiments, the distance not greater than about 2 mm and even not greater than about 1 mm.

In embodiments, the probe of the device is a probe selected from the group consisting of catheters, endoscopes, arthroscopes, amnioscope, colonoscope, pelviscope, bronchoscope, laparscope, esophagogastroduodenoscopes, cystoscopes, ureteroscopes and nephroscopes.

In embodiments, the probe is an aortic catheter, in embodiments between about 1 and 1.5 meter long. In embodiments, the catheter has an outer diameter of less than about 7.26 mm (22 french), less than about 6.6 mm (20 french), and even less than about 5.3 mm (16 french). In embodiments, the catheter has an outer diameter of not less than about 1.98 mm (6 french) and even not less than about 2.64 mm (8 french). Preferably, the outer diameter of the catheter is between about 4.29 mm and 5.3 mm (13-16 french).

In embodiments, the surface of area of the distal tip of the light-guide is not less than about 0.008 mm² (equivalent to a 100 μm diameter circular cross section, not less than about 0.03 mm² (equivalent to a 200 μm diameter circular cross section, and even not less than about 0.126 mm² (equivalent to a 400 μm diameter circular cross section.

In embodiments, the light-guide comprises at least one optical fiber. In embodiments, the optical fiber is of a substantially circular cross section of a diameter of not less than 100 μm, not less than about 200 μm, and even not less than about 400 μm. Preferably, an optical fiber light-guide is between about 400 μm and 600 μm. In embodiments, the light-guide comprises at least two optical fibers. Suitable materials from which to fashion an optical fiber for implementing a light guide of the present invention include, but are not limited to, sapphire, fluoro-aluminate glass and germanium oxide/silica fibers that are suitable for transmission of 2.94 μm light.

In embodiments, the probe further comprises: ii) an irrigation fluid conduit configured to provide a fluid passage from the proximal end of the probe out through a fluid outlet positioned at the distal end of the probe. In embodiments, the distal tip of the probe protrudes a distance from the fluid outlet of the irrigation fluid conduit. In embodiments, the distance not less than about 0.3 mm and even not less than about 0.5 mm. In embodiments, the distance not greater than about 2 mm and even not greater than about 1 mm.

In embodiments, the fluid outlet of the irrigation fluid conduit has an area of not less than about 0.50 mm² (equivalent to a circle with a 0.8 mm diameter), not less than about 1.77 mm² (equivalent to a circle with a 1.5 mm diameter). In embodiments, the fluid outlet has an area of about 2.27 mm² (equivalent to a circle with a 1.7 mm diameter).

In embodiments, a device of the present invention further comprises c) an irrigator functionally associated with the irrigation fluid conduit and configured to introduce liquid into the irrigation fluid conduit from the proximal end and out through the fluid outlet at the distal end. Suitable irrigators include, but are not limited to, pumps, peristaltic pumps and automatically driven syringes. In embodiments, the irrigator is configured to provide intermittent pulses of liquid through the irrigation fluid conduit. Generally, the administration of the irrigation fluid is a part of a cycle that includes an observation step, and optionally a shock wave generation step. Thus the rate of the irrigation fluid pulses varies and is preferably determined by an operator. For example, in embodiments an operator may activate the irrigator to apply regular pulses of irrigation fluid at a frequency of no less than about 1 pulse sec⁻¹ allowing substantially continuous observation of regions of interest while maneuvering the distal tip of the probe. Thus, in embodiments an irrigator is configured to provide intermittent pulses (preferably at a variable rate) of a fluid through the irrigation fluid conduit at a maximal rate that is at least 1 pulse sec⁻¹. In embodiments, the volume of each pulse is not less than about 0.1 ml liquid, and generally not less than about 1 ml liquid. In embodiments, the irrigator is configured to provide a continuous flow of liquid through the irrigation fluid conduit. In embodiments, the irrigator configured to provide a continuous flow of liquid at a rate of no less than about 1 ml min⁻¹, no less than about 2 ml min⁻¹, and even no less than about 5 ml min⁻¹.

In embodiments, the probe further comprises: iii) an aspiration fluid conduit configured to provide a fluid passage from a fluid inlet positioned at the distal end of the probe to the proximal end of the probe. In embodiments, the distal tip of the probe protrudes a distance from the fluid inlet of the aspiration fluid conduit. In embodiments, the distance not less than about 0.3 mm and even not less than about 0.5 mm. In embodiments, the distance not greater than about 2 mm and even not greater than about 1 mm. In embodiments, the fluid inlet of the aspiration fluid conduit has an area of not less than 1.77 mm² (equivalent to a circle with a 1.5 mm diameter), of not less than 2.54 mm² (equivalent to a circle with a 1.8 mm diameter) and even of not less than 3.14 mm² (equivalent to a circle with a 2 mm diameter). In embodiments, the fluid outlet has an area of about 3.8 mm² (equivalent to a circle with a 2.2 mm diameter).

In embodiments, a device of the present invention further comprises d) an aspirator functionally associated with the aspiration fluid conduit and configured to aspirate liquid into the aspiration fluid conduit through the fluid inlet at the distal end of the probe. Suitable aspirators include, but are not limited to, pumps. In embodiments, the aspirator is configured to intermittently aspirate liquid from the fluid inlet at the distal end of the probe. Generally, aspiration is a part of a cycle where an aspiration event is timed to effectively aspirate at least some debris released by generated shock waves. As a result generally, but not necessarily, an aspiration event begins before the first pulse light of a pulse train and ends slightly after the last pulse of light of a pulse train. Thus in embodiments the repetition rate of aspiration events varies and is preferably related to the rate at which the pulse trains of shock-wave generating light are initiated. Since in typical embodiments there is approximately no more than a single train of shock-wave generating light every second, in embodiments, the aspirator is able to intermittently aspirate liquid through the fluid inlet at a maximal frequency that is at least 1 pulse sec⁻¹. In embodiments, each aspiration is of at least 1 ml. In embodiments, the aspirator is configured to provide continuous aspiration of liquid from the fluid inlet at the distal end of the probe. In embodiments, the aspirator is configured to provide aspiration of liquid from the fluid inlet at a rate of at least 12 ml min⁻¹.

In embodiments, a device of the present invention further comprises: e) an illumination component configured to project light out of the proximity of the distal end of the probe where the projected light is of wavelengths and intensity that allows illumination of tissue without damaging the tissue (e.g., between about 200 μm and about 1000 um, such as visible light); and f) an observation component configured to produce an image of an object from light produced by the illumination component and reflected from the object. In embodiments, the distal tip of the probe protrudes a distance from where the illumination component projects light. In embodiments, the distance not less than about 0.3 mm and even not less than about 0.5 mm. In embodiments, the distance not greater than about 2 mm and even not greater than about 1 mm. In embodiments, the distal tip of the probe protrudes a distance from where the observation component acquires reflected light. In embodiments, the distance not less than about 0.3 mm and even not less than about 0.5 mm. In embodiments, the distance not greater than about 2 mm and even not greater than about 1 mm.

In embodiments, the illumination component comprises a light-emitting element located in proximity of the distal end of the probe, such as a light-emitting diode.

In embodiments, the illumination component comprises a light-emitting element functionally associated with the light-guide so that light produced by the light-emitting element is guided through the light-guide out through the distal end of the probe.

In embodiments, a probe of a device of the present invention comprises iv) a second light guide different from the light-guide; and the illumination component which comprises a light-emitting element functionally associated with the second light guide so that light produced by the light-emitting element is guided through the second light guide out through the distal end of the probe.

In embodiments, the illumination component comprises a light-emitting element functionally associated with the irrigation fluid conduit so that light produced by the light-emitting element is guided through an irrigation fluid conduit out through the irrigation fluid outlet proximal to the distal end of the probe. In embodiments, the illumination component comprises a light-emitting element functionally associated with an aspiration fluid conduit so that light produced by the light-emitting element is guided through the aspiration fluid conduit out through the distal end of the probe.

In embodiments, the observation component is functionally associated with the light-guide so as to produce an image of an object from light produced by the illumination component and entering the light-guide through the aspiration inlet proximal to the distal end of the probe.

In embodiments, the probe of a device of the present invention comprises iv) a second light guide different from the light-guide; and the observation component is functionally associated with the second light guide so as to produce an image of an object from light produced by the illumination component and entering the second light guide through the distal end of the probe. In embodiments, the probe of a device of the present invention comprises v) a third light guide, different from the second light guide, configured to guide light between the proximal end and the distal end of the probe; and wherein the illumination component comprises a light-emitting element functionally associated with the third light guide so that light produced by the light-emitting element is guided through the third light guide out through the distal end of the probe.

In embodiments, a probe of a device of the present invention further comprises vi) a cutting tool configured to cut tissue. In embodiments, the cutting tool is a mechanical cutting tool, especially a retractable cutting tool. In embodiments, the cutting tool comprises a cutting light-source light guide configured to guide light between the proximal end of the probe and the distal end of the probe, which can be any of the light guides discussed above (light-guide, second light guide, third light guide) or a dedicated cutting light-source light guide. In embodiments, a device of the present invention, further comprises: g) a light-source functionally associated with the cutting light-source light guide so as to direct light into the cutting light-source light guide from the proximal end of the probe to the distal end of the probe, the light-source configured to provide light suitable for cutting tissue. In embodiments, a distal tip of the cutting light-source light guide is substantially flush with the distal tip of the probe so as to allow cutting tissue which contacts the distal tip of the probe.

In embodiments, a probe of a device of the present invention further comprises vii) an injector, configured to inject a substance into tissue, at the distal end of the probe. In embodiments, the injector comprises a needle, especially a retractable needle. In embodiments, a probe of a device of the present invention further comprises an injectable substance conduit configured to provide a fluid passage from an injectable substance reservoir to the injector.

In embodiments, a device of the present invention further comprises h) a deployable emboli trap positioned about the probe between the distal end and the proximal end of the probe.

In embodiments, a device of the present invention further comprises: i) a heart beat monitor (such as an electrocardiogram device) with an output; and the light-source comprises a trigger functionally associated with the heart beat monitor.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the term “deposits” refers to an unwanted material especially a layer on or within an in vivo tissue and includes, but is not limited to, deposits such as adhesions, calcifications, concretions, deposits, encrustations, plaque, calcified plaque and sediment, whether produced by actual sedimentation or produced by another process.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is an absorption spectrum of water;

FIGS. 2A, 2B and 2C are schematic depictions of a first embodiment of a device of the present invention;

FIG. 3 is a schematic depiction of a device of the present invention used for removing calcified plaque from an aortic valve;

FIGS. 4A and 4B are schematic depictions of a second embodiment of a device of the present invention; and

FIGS. 5A and 5B are schematic depictions of a third embodiment of a device of the present invention.

### DESCRIPTION OF EMBODIMENTS OF THE PRESENT INVENTION

An aspect of the present invention is of methods for removing deposits from in vivo tissue, such as removing calcified plaque from a calcified aortic valve by removing the deposits with the help of shock waves produced by a light-source in a liquid in proximity of the tissue. An aspect of the present invention is of shock wave generating devices that in embodiments are useful in implementing the methods of the present invention.

The principles and operation of the present may be better understood with reference to the drawings and accompanying descriptions. For clarity, an embodiment of a device of the present invention is first described, followed by implementation of an embodiment of the method of the present invention using the device.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The removal of calcified plaque from an aortic valve is no small challenge.

First, calcified plaque comprises hard concretions dispersed in a fibrous matrix. Not only are the hard concretions protected by the fibrous matrix, but the fibrous matrix/hard concretion structure makes the plaque a tenacious, tough and difficult to damage composite material.

Second, the plaque is not an isolated object like a kidney stone, but rather is a tenacious layer of fibrous material with embedded calcifications that covers and strongly adheres to a tissue that needs to be treated, the aortic cusps.

Third, aortic cusps are exceedingly delicate such that physical or thermal damage often leads to scarring, perforation, occlusion, burn damage, cusp retraction and/or thickening, adversely affecting the valve function. Exposure to ultrasound has been demonstrated to destroy aortic cusps including by delamination of the cusps without effective removal of plaque (Williamson W A, Aretz H T, Weng G, Shahian D M, Hamilton W M, Pankratov M M, Shapshay in Light-sources Surg Med. 1993, 13(4), 421-428).

Prior art shock wave generating laser lithotripters use relatively high-powered pulses of light-source light at wavelengths that are used to destroy isolated concretions such as kidney stones. The wavelengths of the light generated by such lithotripters are selected such that the light is absorbed to some degree both by the concretions and by the aqueous liquid surrounding the concretions so that the light pulses destroy the concretions by a combination of mechanisms. When a prior art shock wave generating laser lithotriptor is activated, at least some light energy is absorbed by the liquid present between the light guide and the concretion, presumably generating a shock wave in the liquid which usually pushes the concretion away. It is not clear if a significant amount of energy of the shock wave produced in the liquid is transferred into the concretion to destroy. The light absorbed by the concretion destroys the concretion by a combination of direct thermal ablation, heating and photoacoustic shock waves generated in the concretion itself. Prior art shock wave generating laser lithotripters are ineffective and unsafe for use to remove deposits covering a tissue such as calcified plaque on an aortic cusp. As noted above, the fibrous component of calcified plaque protects the embedded concretions from exposure to the light generated by the lithotripters. The laser light cuts and denaturizes, but does not disintegrate, the fibrous component. Not only does the released heat damage the aortic valve, but the light is ineffective in separating the fibrous matrix from the cusp. Even if a concretion is exposed and irradiated with the lithotripter light, the fragments caused by explosion of the concretion, the heat generated in the surrounding liquid and in the concretion as well as lithotripter light penetrating past the concretion into the tissue of the aortic valve all cause catastrophic damage to the cusps.

The prior art also does not teach how to perform medical procedures with the help of laser-generated shock waves on a beating heart or in the presence of significant blood flow, flow that reduces visibility and carries away concretion fragments.

The present invention is of methods and devices for removing deposits from in vivo tissue, such as removing calcified plaque from a calcified aortic valve, by removing the deposits with the help of shock waves produced in a liquid by a light-source. Embodiments of the present invention are suitable for use in the presence of high liquid flow past the tissue and/or under poor-visibility conditions.

The effectiveness of embodiments of the present invention in removing deposits from in vivo tissue is based, at least in part, on generating a pulse train of light having parameters that produce almost exclusively shock waves in a surrounding liquid with substantially no release of heat, the produced shock waves impacting the deposits with sufficient energy and frequency to disintegrate and dislodge the deposits, layer by layer, preferably without damaging the underlying tissue. Surprisingly, it has been found that when the correct parameters of the pulse trains of light are selected, the shock waves produced in the liquid are sufficient to transfer sufficient energy into the calcified plaque and thus to disintegrate this tenacious composite material with substantially no damage to the underlying tissue.

In the present invention, the distal tip of a light-guide (preferably mounted on a probe such as a catheter or an endoscope) is brought to an effective distance from a tissue to be treated. In embodiments, a typical effective distance is greater than 0.3 mm or even greater than about 0.5 mm from the tissue and generally less than about 2 mm or even less than about 1 mm from the tissue.

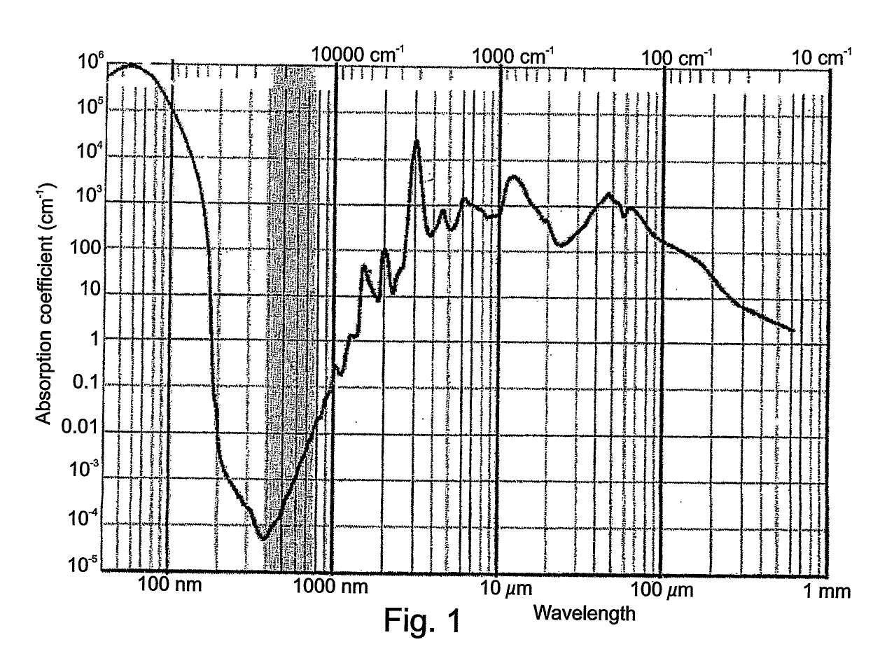

A light-source, functionally associated with the light-guide so as to direct light into the light-guide, is configured to generate a pulse train which comprises at least one pulse of light, each pulse of light of an energy, of a duration less than about 300 ns and including at least one wavelength of light such as to generate a shock wave in an aqueous liquid (e.g., blood or saline) wherein at least one wavelength is a wavelength having a water absorption coefficient of no less than about 10³ cm⁻¹ and preferably of no less than 10⁴ cm⁻¹. As seen from FIG. 1, suitable wavelengths of light include wavelengths between about 2.8 μm and 3.5 μm, between about 5.9 μm and 6.4 μm and between about 12 μm and 18 μm, but preferably between about 2.6 μm and about 3.1 μm. In embodiments, at least one wavelength produced by the light-source is 2.94 μm which, as seen in FIG. 1, is a wavelength having close to maximal absorption in water.

Wavelengths of light having a water absorption coefficient of no less than about 10³ cm⁻¹ are substantially entirely absorbed by water. The energy of the light is almost entirely converted into heat, which if sufficient, superheats the water, leading to the explosive formation of a bubble of water vapor producing an expansion shock wave, and under certain conditions, also a subsequent cavitation shock wave.

If the pulse duration is too long, e.g., longer than about 300 ns, there is a chance that at least some of the absorbed energy will dissipate into the surrounding bulk water, heating the water. Further, if the light is not absorbed by vapor, the light may pass through the vapor bubble and be absorbed by what is behind the bubble, e.g. sensitive tissue. Further, shock waves are most efficiently produced when the light is absorbed by a constant volume of water (an isochoric process). With a sufficiently short pulse when there is no time for heat dissipation by the water and no time for expansion of the water so that the maximal local superheating is achieved, it is possible to achieve near instantaneous vaporization of water, providing the theoretical pressure limit of 225 atmospheres so as to efficiently produce an expansion shock wave. Thus, in embodiments, the duration of the pulses of light is less than about 300 ns, less than about 200 ns, less than about 100 ns, less than 50 ns and even less than 10 ns.

In embodiments, substantially all wavelengths of light guided through the light-guide have a water absorption coefficient of no less than about 10³ cm⁻¹, e.g., the light-source produces only such light or there is a filter or the like that prevents such from passing through the light-guide. In such a way, frequencies of light that are not absorbed by water are prevented from penetrating the water and damaging the treated tissue.

In order to efficiently degrade calcified plaque, it is necessary that the generated shock waves be sufficiently energetic. Thus in embodiments, the pulses of light generated by the light-source and guided through the light guide emerge from the distal tip of the light-guide at an energy of at least about 1 mJ, at least about 2 mJ, and even at least about 5 mJ. That said, in embodiments of the present invention, the energy emerging from the distal tip of the light-guide is less than about 1000 mJ, less than about 500 mJ and even less than about 100 mJ.

In order to sufficiently degrade calcified plaque at a reasonable rate, it is often necessary to expose the same area to multiple shock waves. Thus, in embodiments, the generated pulse train comprises not less than two pulses of light at a frequency of not lower than 2 Hz. In embodiments the pulse train comprises not less than five pulses of light. In embodiments, the frequency is not lower than 5 Hz and even not lower than 10 Hz. In order to avoid ultrasound damage to the cusps, in embodiments the frequency is not higher than 1000 Hz, not higher than 500 Hz, not higher than 250 Hz and even not higher than 100 Hz.

Commercially available light-sources that are suitable for producing light pulses with the properties for implementing the teachings of the present invention include lasers, such as Q-switched Er:YAG lasers configured to produce pulses of 2.94 μm light having a duration of between about 100 and 200 ns and Nd:YAG laser-pumped OPOs configured to produce pulses of 2.94 μm light having a duration of between about 1 and 15 ns.

As the light energy is substantially entirely absorbed in the immediate vicinity of the distal tip of the light-guide, in embodiments it is important that the surface area of the distal tip of the light-guide be sufficiently large so that a sufficient volume of water be vaporized. Thus in embodiments, the interface of the light-guide and the aqueous liquid (e.g. blood, saline) is not less than about 0.008 mm² (substantially equivalent to that of a 100 μm diameter circle), not less than about 0.03 mm² (substantially equivalent to that of a 200 μm diameter circle), and even not less than about 0.126 mm² (substantially equivalent to that of a 400 μm diameter circle).

The present invention is most easily understood with reference to an embodiment of the method of the present invention and a specific embodiment of a device of the present invention.

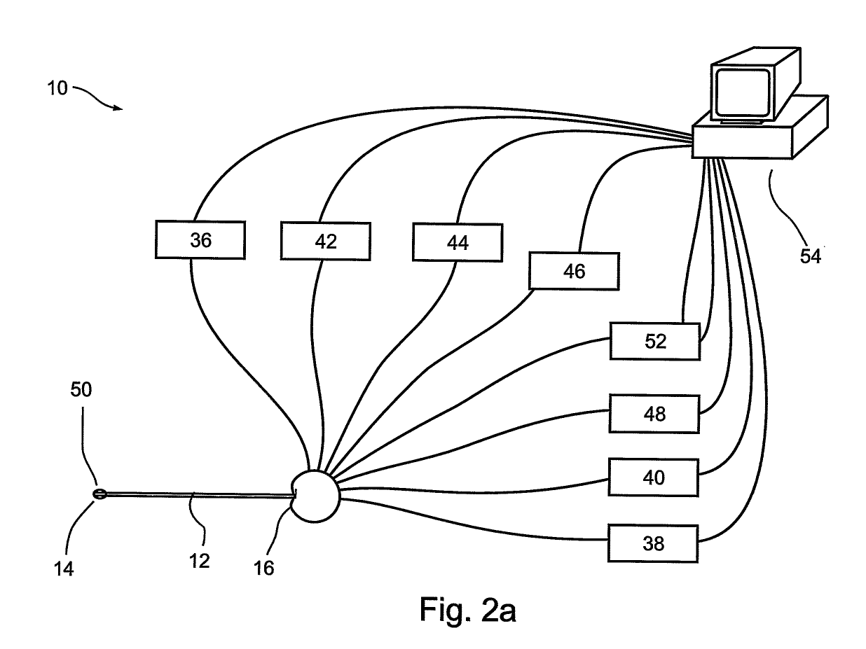

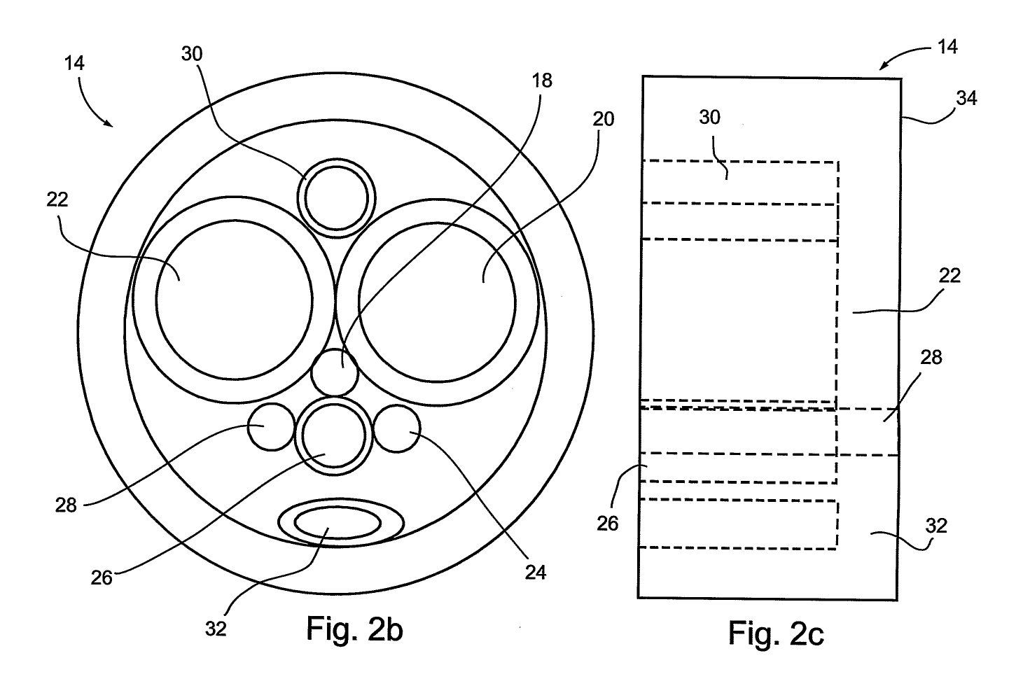

In FIGS. 2A, 2B and 2C is depicted a device 10 of the present invention.

In FIG. 2A, device 10 is schematically depicted and includes a cardiac catheter 12 (20 french/6.6 mm diameter, 1.5 meter long) as a probe with a distal end 14 and a proximal end 16.

In FIG. 2B, distal end 14 of catheter 12 is depicted head-on so that the distal tips of eight parallel, not coaxial, channels are seen:

a light-guide 18 (a 425 μm sapphire fiber optic with 63 μm cladding) with a surface area at the distal tip of 0.14 mm²;

an irrigation fluid conduit 20, a 2 mm outer diameter, 1.4 mm inner diameter PEEK tube with 0.3 mm thick walls, the inner diameter of the tube at the distal tip defining a fluid outlet having an area of 1.54 mm²;

an aspiration fluid conduit 22, a 2 mm outer diameter, 1.4 mm inner diameter PEEK tube with 0.3 mm thick walls, the inner diameter of the tube at the distal tip defining a fluid inlet having an area of 1.54 mm²;

an illumination light guide 24, a 425 μm sapphire fiber optic with 63 μm cladding, provided with a dispersing lens so that light exiting illumination light guide 24 illuminates a relatively large area 1 mm from the distal tip of illumination light guide 24;

an observation light guide 26, a 0.6 mm diameter fiber optic cable with 0.2 mm thick cladding suitable for acquiring a pixelated image, positioned so as to acquire light exiting the distal tip of illumination light guide 24 and reflected from objects 1 mm therefrom;

a cutting light-source light guide 28, a 425 μm sapphire fiber optic with 63 μm cladding, configured to guide tissue-cutting light from the proximal end of cutting light-source light guide 28 out through the distal tip of cutting light-source light guide 28;

an injector guide tube 30, a 1 mm outer diameter, 0.6 mm inner diameter PEEK tube with 0.2 mm thick walls, configured to slidingly contain a retractable hollow injector needle (not depicted); and

a guide wire channel 32, a standard catheter guide wire channel for use with ribbon guide wires up to 2 mm wide and 0.6 mm thick.

In FIG. 2C, distal end 14 of catheter 12 is depicted in side view with some internal features drawn with dashed lines. It is seen that distal tip 34 of catheter 12 protrudes 1 mm from the distal ends of light-guide 18, irrigation fluid conduit 20, aspiration fluid conduit 22, illumination light guide 24, observation light guide 26, injector guide tube 30 and guide wire channel 32 which are all substantially flush with each other. Only cutting light-source light guide 28 is flush with distal tip 34 of catheter 12.

In FIG. 2A, peripheral components of device 10 are depicted, functionally associated with the appropriate channels of catheter 12 through proximal end 16 of catheter 12.

Shock wave generating light-source 36 is an OPO Nd:YAG light-source (Blue Sky Research, Milpitas, Calif., USA) configured to produce pulses of 2.94 μm light having a duration of between about 1 and 15 ns at up to 10 Hz and is functionally associated with light-guide 18 so that light generated by light-source 36 is guided from the proximal end of light-guide 18 to emerge from the distal tip of light-guide 18.

Irrigator 38 is, for example, a commercially available pump with an adjustable flow rate (whether intermittently or continuous) provided with a reservoir holding saline. Irrigator 38 is functionally associated with irrigation fluid conduit 20 and is configured to introduce the saline held in the reservoir through proximal end 16 of catheter 12 and out through the fluid outlet defined as the distal tip of irrigation fluid conduit 20.

An aspirator 40 is, for example, a commercially available pump with an adjustable aspiration rate (whether intermittently or continuous). Aspirator 40 is functionally associated with aspiration fluid conduit 22 and is configured to apply suction from the proximal end of aspiration fluid conduit 22 so as to aspirate liquid into the fluid inlet defined as the distal tip of aspiration fluid conduit 22.

An illumination component 42 is a commercially available visible light source suitable for use with fiber optic light guides such as illumination light guide 24, with which illumination component 42 is functionally associated through the proximal end of illumination light guide 24. When illumination component 42 is activated, light from illumination component 42 enters the proximal end of illumination light guide 24 and is guided thereby to emerge from the distal tip of illumination light guide 24 to illuminate areas at which distal end 14 of catheter 12 is pointed.

An observation component 44 is, for example, a commercially available CCD light detector for use with fiber optic cable light guides such as observation light guide 26 with which observation component 44 is functionally associated through the proximal end of observation light guide 26. Observation component 44 includes the appropriate hardware and software to generate and display an image from light acquired through observation light guide 26.

A cutting light-source 46 is functionally associated with cutting light-source light guide 28 to direct light into cutting light-source light guide 28 from the proximal end of cutting light-source light guide 28. In embodiments, a cutting light-source is a dedicated light source known in the art of surgery, such as an Argon ion laser (Spectra-Physics, Mountain View, Calif., USA). In embodiments, a cutting light-source is the same light source used for generating shock-waves, which is optionally operated at a wavelength different from the wavelength used to generate shock waves.

An injector 48 comprises a hollow injector needle, constituting an injectable substance conduit slidingly contained in injector guide tube 30 and configured to extend outwards up to 3 mm. Injector 48 also comprises a liquid reservoir (holding, e.g., a liquid API (active pharmaceutical ingredient) composition such as Pacxitacel) and is configured to drive a liquid held in the reservoir through the hollow injector needle.

A deployable emboli trap 50 is positioned about the shaft of catheter 12 between distal end 14 and proximal end 16 but close to distal end. Deployable emboli trap 50 is substantially analogous to emboli traps known in the art such as EmboShield (Abbott Laboratories, Abbott Park, Ill., USA), RX Accunet (Guidant Corporation, Indianapolis, Ind., USA), AngioGuard XP (Cordis Corporation, Miami Lakes, Fla., USA) or Filterwire EX (Boston Scientific Corporation, Natick, Mass., USA). In a non-deployed position emboli trap 50 is pressed against catheter 12. In a deployed state, emboli trap 50 opens outwards.

A heart beat monitor 52 is a prior art electrocardiogram device with an output that is functionally associated with the trigger of shock wave producing light-source 36.

A controller 54 is functionally associated with the other peripheral components of device 10 so as to be able to monitor and control operation of the peripheral components.

Device 10 can be used in implementing the method of the present invention. Device 10 is readied for use, for example by attaching the peripheral components to catheter 12.

Parameters to generate a pulse train to produce effective shock wave are selected for shock wave generating light-source 36 including wavelength (e.g., 2.94 μm), number of pulses (e.g., 5 pulses), duration of each pulse (e.g., 10 ns), energy of each pulse (e.g., 10 mJ as measured at the proximal tip of light-guide 18) and frequency of the pulse train (e.g., 10 Hz).

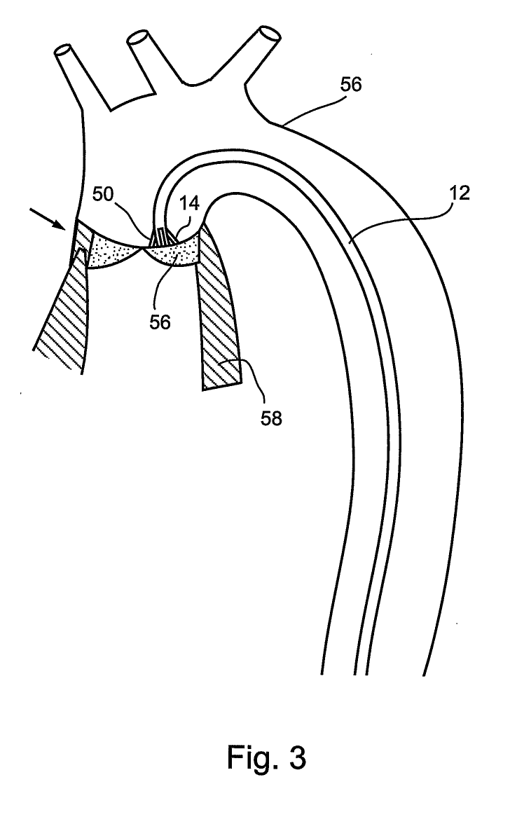

Distal end 14 of catheter 12 is brought, in the usual way, through an aorta 56 to the proximity of an area to be treated, in FIG. 3 a calcified aortic valve 56 of a heart 58.

Emboli trap 50 is deployed so as to potentially capture at least some dislodged plaque.

Illumination component 42 and observation component 44 are activated to allow observation of the area of aortic valve 56 in proximity with distal end 14 of catheter 12. Irrigator 38 is activated to provide a continuous flow of saline out through the outlet of irrigation fluid conduit 20. The saline flow displaces blood located at distal end 14 of catheter 12 allowing light from illumination component 42 exiting illumination light guide 24 to illuminate areas of aortic valve 56 and allowing the light to enter observation light guide 26 to be detected by observation component 44. The rate and volume of the saline flow is adjusted to provide a relatively continuous, relatively clear image of areas of aortic valve, in embodiments no less than about 1 ml min⁻¹, no less than about 2 ml min⁻¹, and even no less than about 5 ml min⁻¹.

An operator examines aortic valve 56 and selects an area of aortic valve 56 for treatment. Once an area is selected, the distal tip of light-guide 18 is brought to an effective and safe distance from the area. As distal tip 34 of catheter 10 protrudes from the distal tip of light-guide 18 by 1 mm, simply pressing distal tip 34 of catheter 10 against an area to be treated ensures that an effective and safe distance is maintained.

The operator activates controller 54 to initiate a shock wave producing event. Controller 54 activates aspirator 40 to begin an 800 millisecond aspiration event of fluids through the inlet of aspiration fluid conduit 22. Controller 54 monitors the output of heart beat monitor 52, for example the R-wave of the ECG. At systole, and no sooner than 100 milliseconds after initiation of aspiration (to ensure that there is sufficient aspiration), controller 54 activates shock wave producing light-source 36 to generate light that is guided by light-guide 18 to proximity of the treated area.

When activated, shock wave generating light-source 36 produces a 400 millisecond pulse train of light according to the selected parameters, as noted above: five 10 ns pulses of 2.94 μm light at 10 Hz with an energy of 10 mJ at the distal tip of light-guide 18. As the wavelength used has a water absorption coefficient of more than about 10⁴ cm⁻¹, substantially all light exiting light-guide 18 is absorbed in the water in the immediate proximity of the distal tip of light-guide 18. As the pulse duration is short and as the distal tip of light-guide 18 is maintained at a 1 mm distance from the tissue being treated, there is substantially no penetration of light past the immediate proximity of the distal tip of light-guide 18 so there is no fear that light energy will impact the treated tissue. Further, each of the five pulses is sufficiently energetic to generate an expansion shock wave, and under certain conditions, also a subsequent cavitation shock wave, that dislodges or weakens at least some of the plaque on aortic valve 56. The frequency of 10 Hz is such that a succeeding pulse of light is initiated after complete collapse of a bubble produced by a preceding pulse of light.

Aspirator 40 is set to aspirate at a rate that is sufficient to absorb a significant proportion of the dislodged plaque, in embodiments, each aspiration of at least 1 ml over the 800 millisecond aspiration event. Since aspirator 40 is activated for 800 milliseconds, throughout the 400 millisecond pulse train and attendant shock wave event, aspiration of liquid and dislodged plaque is aspirated into the inlet of aspiration fluid conduit 22.

At least some dislodged plaque that is not aspirated is captured by deployed emboli trap 50.

The above procedure is repeated for different areas of aortic valve 56 and if necessary repeated multiple times for any given area, until sufficient calcified plaque is removed from the aortic valve. It is important to note that it is not clinically necessary to remove all of the calcified plaque, but just sufficient plaque to improve aortic valve functioning.

It is known that calcific aortic stenosis often leads to fusion of the cusp commissures. During the above procedure, and optionally at the end of the procedure after sufficient plaque has been removed, the commissural fusion is optionally released by cutting, for example using light produced by cutting light-source 46 and projected through cutting light-source light guide 28 under guidance of an operator using observation component 44 and illumination component 42.

During the above procedure, and preferably at the end of the procedure, a liquid such as a liquid API composition is optionally administered to the treated area, for example through injector guide tube 30 with the help of injector 48. An example of a suitable API to be administered is an anti-scarring agent such as Pacxitacel for example by injection to a cusp of aortic valve 56.

The method of the present invention is described above where the firing of shock wave generating light-source 36 to generate shock waves was in part triggered by input from heart beat monitor 52. In embodiments, firing of a light-source 36 to generate shock waves is triggered substantially entirely by the operator and is not dependent on monitoring the heart beat of the subject being treated. In such embodiments, a heart 58 to be treated is optionally paced to increase the heart beat, typically to at least about 100 beats per minute, at least about 180 beats per minute and even at least about 200 beats per minute. During pacing, the stroke of an aortic valve 56 is small so there is relatively little motion of the cusps of the aortic valve. It is thus simpler to ensure that distal tip 34 of catheter 10 is in contact with a cusp of aortic valve 56 while shock waves are being generated.

The method of the present invention is described above where there is a continuous flow of saline from irrigation fluid conduit 20 so that it is possible to continuously observe the area being treated. In some cases such a continuous flow of saline may raise concerns that too much saline is administered to the subject. In embodiments, irrigator 38 is activated intermittently to provide pulses of saline periodically or only upon demand (e.g. for displacing blood to allow optical observation) when triggered by an operator through controller 54. In embodiments, the volume of each such is not less than about 0.1 ml liquid, and generally not less than about 1 ml liquid.

The method of the present invention was described above where there is only an intermittent aspiration by aspirator 40 through aspiration fluid conduit 22 to reduce the amount of fluids removed from the subject. In embodiments, aspirator 40 is activated continuously. In embodiments, an aspirator 40 aspirates fluid at a rate of not less than 12 ml min⁻¹.

The method of the present invention was described above where observation component 44 and illumination component 42 are continuously activated. In embodiments, observation component 44 and illumination component 42 are activated intermittently.

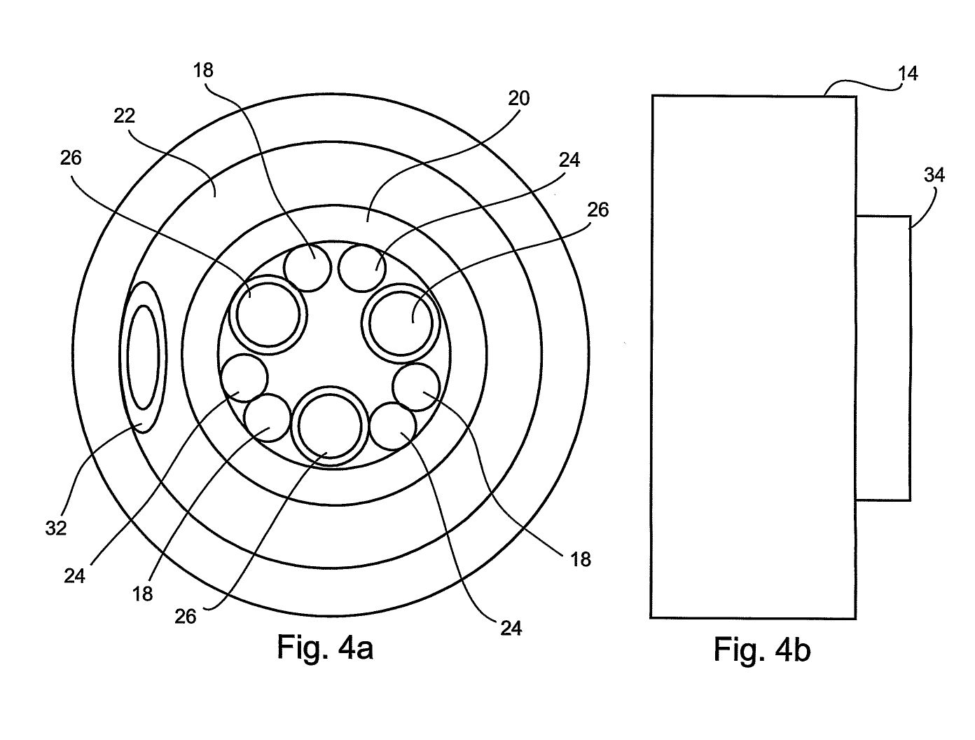

An alternative embodiment of a probe of the present invention, catheter 62 is depicted in FIG. 4A (depicted head-on) and in FIG. 4B (depicted in side view).

Structurally, catheter 62 is substantially similar to catheter 12 depicted in FIGS. 2A, 2B and 2C with a few significant differences. In FIG. 4A are seen the distal tips of twelve parallel channels. Three light-guide channels 18, three illumination light probe channels 20 and three observation light probe channels 26 are all contained inside the bore of an irrigation fluid conduit 20 (a 4 mm outer diameter, 3 mm inner diameter PEEK tube). Irrigation fluid conduit 20 is contained inside and coaxial with the bore of aspiration fluid conduit 22 (defined by the outer wall of catheter 62 a 20 french 6.6 mm outer diameter, 5.5 mm inner diameter PEEK tube). Guide wire channel 32 is also contained within the bore of aspiration fluid conduit 22. Catheter 62 is devoid of a cutting light-source probe and an injector probe tube.

In FIG. 4B, distal end 14 of catheter 62 is depicted in side view. The distal tip of irrigation fluid conduit 20 protrudes 1 mm from the distal ends of light guide channels 18, aspiration fluid conduit 22, illumination light guides 24 and observation light guides 26 and therefore defines distal tip 34 of catheter 62.

The use of catheter 62 is, in analogy to the use of catheter 12 depicted in FIGS. 2A, 2B and 2C as discussed above, clear to one skilled in the art upon perusal of the description herein.

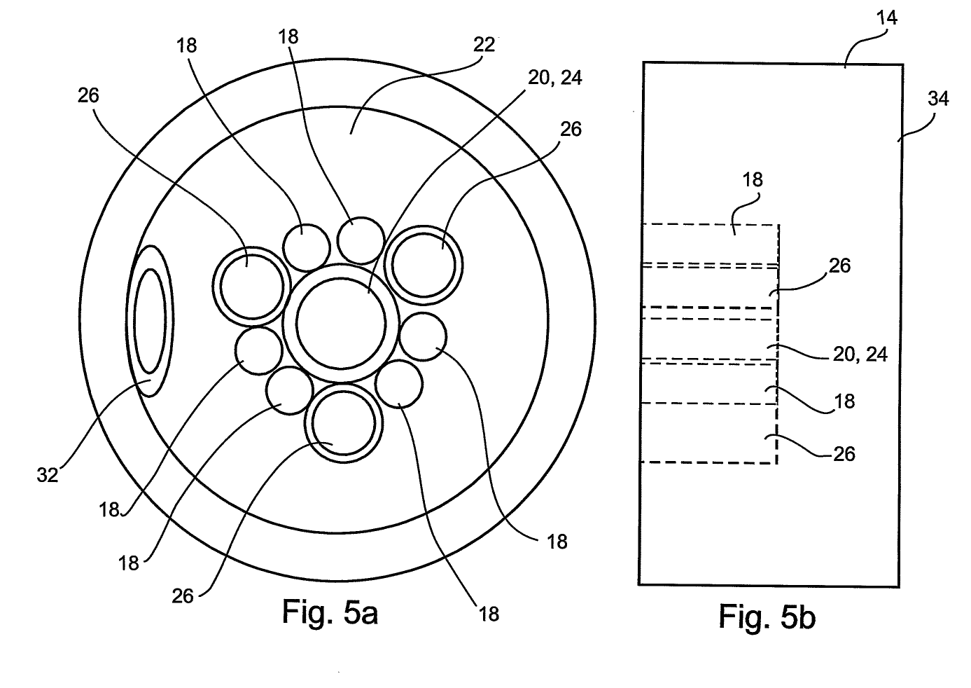

An alternative embodiment of a probe of the present invention, catheter 64 is depicted in FIG. 5A (depicted head-on) and in FIG. 5B (depicted in side view).

Structurally, catheter 64 is substantially similar to catheter 12 depicted in FIGS. 2A, 2B and 2C with a few significant differences. In FIG. 5A are seen the distal tips of twelve parallel channels. Six light-guide channels 18 and three observation light guide channels 26 all surround an irrigation fluid conduit 20 (a 3 mm outer diameter, 2.2 mm inner diameter PEEK tube). The inner lumen of irrigation fluid conduit 20 is coated with a reflective coating so that irrigation fluid conduit also functions as an illumination light guide 24. The above channels are contained inside (and irrigation fluid conduit 20 also coaxial with) the bore of aspiration fluid conduit 22 (defined by the outer wall of catheter 64, a 20 french 6.6 mm outer diameter, 5.5 mm inner diameter PEEK tube). Guide wire channel 32 is also contained within the bore of aspiration fluid conduit 22. Catheter 64 is devoid of a cutting light-source guide and an injector guide tube.

In FIG. 5B, distal end 14 of catheter 12 is depicted in side view with some internal features drawn with dashed lines. It is seen that distal tip 34 of catheter 12 protrudes 1 mm from the distal ends of light-guide 18, irrigation fluid conduit 20/illumination light guide 24, observation light guides 26 and guide wire channel 32 which are all substantially flush with each other.

The use of catheter 64 is, in analogy to the use of catheter 12 depicted in FIGS. 2A, 2B and 2C as discussed above, clear to one skilled in the art upon perusal of the description herein.

In the depicted embodiments, an illumination light guide 24 is a dedicated illumination light guide 24 or an irrigation fluid conduit 20. In embodiments, an illumination light guide 24 comprises a light-guide 18 or an aspiration fluid conduit 22.

In the depicted embodiments, an illumination component 42 of a device of the present invention is a peripheral component that illuminates an area in proximity of the distal tip of a catheter through an illumination light guide 24. In embodiments, an illumination component comprises a light-emitting element (such as a light-emitting diode) located in proximity of the distal end of the probe.

In the depicted embodiments, an observation component 44 of a device of the present invention is functionally associated with a dedicated observation light guide or light guides 26. In embodiments, an observation component 44 of a device of the present invention is functionally associated with a light-guide 18.

In the depicted embodiments, a cutting tool comprises a cutting light-source 46 and a dedicated cutting light-source light guide 28 to guide light from cutting light-source 46 to the locus to be cut. In embodiments, light from a cutting light-source is guided through a non-dedicated light guide, e.g., a light-guide 18, an illumination light guide 24 or an observation light guide 26.

### EXPERIMENTAL

Reference is now made to the following example which together with the above description illustrates the invention in a non-limiting fashion.

An Nd:YAG laser-pumped OPO (Blue Sky Research, Milpitas, Calif., USA) was set to direct 10 mJ pulses of 10 ns duration of 2.94 μm light into the proximal end of an 80 cm long sapphire fiber with a diameter of 0.425 mm. 5 mJ of light emerged from the distal tip of the fiber.

The distal tip of the fiber was immersed in a saline solution and the generation of bubbles as a result of superheating of water at the distal tip/saline interface was observed with flash photography.

It was seen that each light-source pulse initiated an event that lasted less than about 500 μs.

Approximately 1 μs after a 10 ns light-source pulse a first, almost hemispherical, shock wave was observed moving from the distal tip of the fiber.

Approximately 2 μs after a 10 ns light-source pulse the shockwave was observed to expand while a small bubble was observed at the distal tip of the fiber.

Approximately 90 μs after a 10 ns light-source pulse the shockwave had disappeared from the field of view but the bubble was observed at to be approximately 4-5 mm in diameter and began to detach and move away from the distal tip of the fiber.

Approximately 150 μs after a 10 ns light-source pulse the bubble was observed to have already collapsed and a cavitation shock wave was observed.

Approximately 400-500 μs after a 10 ns light-source pulse the saline bath was still and the event initiated by the light-source pulse had ended.

It is expected that during the life of this patent many relevant technologies and materials will be developed and the scope of the terms used herein is intended to include all such new technologies and materials a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Although the present invention was described primarily in the context of removing calcified plaque from an aortic valve, the methods and devices of the present invention may be modified for treating other indications in various locations in the body.

In cardiovascular applications the teachings of the present invention may be applied to removing plaque and other deposits from the luminal surface of a vein, a luminal surface of an artery, a heart valve, an aortic valve, and a valve seat, especially prior to implantation of an artificial valve or alternate valve as well as for treating indications including but not limited to saddle thrombosis bifurcations or for clogged stent decalcification.

In orthopedic applications the teachings of the present invention may be applied to treating indications including but not limited to gouty arthritis, deposits resulting from hyperuricemia, periostitis, exostosis, bone growth following spinal surgery and hydroxiapatite or other deposits of the wrist.

The teachings of the present invention may also be applied to removal of deposits from the vocal cords.

The teachings of the present invention may also be applied to treat conditions that are treated using prior-art light-source lithotripsy procedures such as treatment of stones in the gall bladder or passages and kidney stones.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A shock-wave generating device for invivo removal of deposits from tissue, comprising:
a) a probe with a proximal end and a distal end, said distal end having a distal tip, said probe comprises:
i) a light-guide configured to guide shock wave-generating light to the distal end of said probe;
b) a light-source directing light into said light-guide, said light-source configured to generate a pulse train which comprises at least one pulse of light, each said pulse of light of a duration of less than 300 ns, which generates a shock wave in an aqueous liquid wherein at least one wavelength of said light is a wavelength having a water absorption coefficient of no less than about 10³ cm⁻¹.

2. The device of claim 1 wherein said wavelength is between 2.8 μm and 3.5 μm.

3. The device of claim 1, wherein said duration of said pulse of light is 15 ns or less.

4. The device of claim 1, wherein said pulse train consists of not less than two pulses of light at a frequency of not lower than 2 Hz.

5-7. (canceled)

8. The device of claim 1, wherein said probe is an aortic catheter.

9-11. (canceled)

12. The device of claim 1, said probe further comprising:
an irrigation fluid conduit, configured to provide a fluid passage from a proximal end of said probe out through a fluid outlet positioned at a distal end of said probe.

13. (canceled)

14. The device of claim 1, said probe further comprises:
iii) an aspiration fluid conduit configured to provide a fluid passage from a fluid inlet positioned at a distal end of said probe to a proximal end of said probe.

15. (canceled)

16. The device of claim 1, further comprising:
e) an illumination component configured to project light out of a projecting port at proximity of said distal end of said probe; and
f) an observation component configured to acquire, light produced by said illumination component and received at the observation component from an object and produce an image of the object.

17-18. (canceled)

19. The device of claim 1, said probe further comprising:
vi) a cutting tool configured to cut tissue.

20. The device of claim 19, wherein said cutting tool comprises a cutting light-source light guide configured to guide light between said proximal end of said probe and said distal end of said probe.

21. The device of claim 1, further comprising:
i) a heart beat monitor with an output;
and wherein
said light-source comprises a trigger functionally associated with said heart beat monitor.

22. A method of dislodging plaque from an aortic valve, the method comprising:
a) providing a cardiac catheter having a distal end and a distal tip, which comprises a light-guide, a distal tip of said light-guide in proximity of a distal end of said catheter;
b) placing said distal tip of said light-guide at a distance from a calcified area of said aortic valve;
c) guiding a pulse train which comprises at least one pulse of light, each pulse of light of an energy, of a duration and including at least one wavelength of light such as to generate a shock wave in said aqueous liquid at an interface between said distal tip of said light-guide and said aqueous liquid, said pulse train sufficient to dislodge at least some plaque coating from said area,
wherein at least one said wavelength is a wavelength having a water absorption coefficient of no less than about 10³ cm⁻¹ and
wherein said duration is less than about 300 ns.

23. The method of claim 22, wherein said heart is beating.

24. The method of claim 23, wherein generation of said pulse train of light is coordinated with said beating of said heart.

25. The method of claim 22, wherein said distal tip of said catheter protrudes substantially said distance from said distal tip of said light-guide and said placing said distal tip of said light-guide at a distance from said area comprises contacting said distal tip of said catheter against said aortic valve in proximity of said area.

26-27. (canceled)

28. The method of claim 22, wherein said distance is not greater than about 2 mm.

29. The method of claim 22, wherein said distance is not less than about 0.3 mm.

30. The method of claim 22, wherein substantially all wavelengths of light guided through said light-guide have a water absorption coefficient of no less than about 10³ cm⁻¹.

31. The method of claim 22, wherein said duration of said pulse of light is less than 15 ns.

32-33. (canceled)

34. The method of claim 22, wherein said frequency is not lower than about 5 Hz.

35. The method claim 22, wherein said frequency is not higher than about 1000 Hz.

36. The method of claim 22, further comprising:
iv) prior to guiding a pulse train, observing said area.

37. The method of claim 36, wherein said observing said area comprises optically observing said area.

38. The method of claim 37, further comprising, during said optical observation, illuminating said area with at least one wavelength of light substantially not absorbed by said aqueous liquid.

39. The method of claim 37, further comprising:
v) prior to said optical observing, irrigating said area with a substantially transparent liquid so as to displace said aqueous liquid to allow clearer said observation of said area.

40. The method of claim 22, further comprising:
vi) aspirating at least some of said dislodged deposits.

41. The method of claim 40, wherein said aspiration is intermittent.

42. The method of claim 40, wherein said aspiration occurs during said guiding of said pulse train of said light.

43. The method of claim 22, further comprising:
d) cutting commissural fusion of said calcified aortic valve.

44. The method of claim 43, wherein said cutting is with light projected through a light guide associated with said catheter.

45. The device of claim 1, wherein said catheter has a distal tip which protrudes a distance from at least one of the following:
a distal tip of said light-guide;
said fluid outlet of said irrigation fluid conduit;
said fluid inlet of said aspiration fluid conduit;
said projecting port;
where said observation component acquires light.

46. The device of claim 45, wherein said distance is between 0.3 and 2 mm.

47. The device of claim 45, wherein the surface area of the protruding distal tip of said light-guide is not less than about 0.008 mm².
